(19) United States
(12) Patent Application Publication
(10) Pub. No.: US 2023/0320682 A1
(43) Pub. Date: Oct. 12, 2023

(54) MEANS AND METHODS FOR IMPROVED CORONARY INTERVENTIONS

(71) Applicant: CoreAalst BV, Aalst (BE)

(72) Inventors: Jeroen Sonck, Wemmel (BE); Carlos Collet, Herne (BE); Dimitri Buytaert, Gent (BE)

(21) Appl. No.: 17/923,196

(22) PCT Filed: May 7, 2021
(86) PCT No.: PCT/EP2021/062152

§ 371 Date: Nov. 3, 2022

(30) Foreign Application Priority Data
May 7, 2020 (GB) 2006750.0

(51) Int. Cl. A61B 6/00

(52) U.S. Cl. CPC A61B 6/504; A61B 6/466; A61B 6/56; A61B 6/547; A61B 6/4441

(57) ABSTRACT

The present invention provides means and methods for the assessment of coronary vessels, in particular to determine the patterns of blockage or restriction to the blood flow through a coronary vessel. More particularly, the present invention relates to a generalized approach to synchronize angiographic projection angles in real time with external 3-dimensional coronary CT angiographic images. Accordingly, a system is provided to project two-dimensional angiographic images side by side with three-dimensional coronary CT images.

### FIELD OF THE INVENTION

The present invention relates to the field of cardiac disease, in particular to the assessment of coronary vessels, in particular to determine the patterns of blockage or restriction to the blood flow through a coronary vessel. More particularly, the present invention relates to a generalized approach to synchronize angiographic projection angles in real time with external 3-dimensional coronary CT angiographic images leading to a visualization of the atherosclerotic plaque and its components during conventional angiographic procedures. The present invention provides systems and devices to project two-dimensional angiographic images side by side with three-dimensional coronary CT images.

### INTRODUCTION TO THE INVENTION

Over the last two decades, computed tomography (CT) has become an established tool in the diagnostic work up of patients with suspected coronary artery disease (CAD). Non-invasive imaging of coronary arteries by CT angiography (CCTA) allows detecting atherosclerotic disease (not visible with conventional angiography) and, assessing for the presence of obstructive disease and risk stratify patients based on plaque characteristics. (1) (2). Current guidelines emphasize the role of CCTA as a first line test for patients with symptoms suggestive of obstructive CAD. (3) Nevertheless, CCTA remains a diagnostic tool, its usefulness beyond this phase has not been yet fully explored. Accordingly, there is a need to develop the potential of CCTA to help plan and guide coronary interventions in a fashion similar to how cardiac CTA has transformed transcatheter heart valve interventions. There exists also a need to implement CCTA in the cath-lab to guide coronary interventions.

### SUMMARY OF THE INVENTION

According to an aspect of the invention, there is provided a system configured to display three-dimensional (3D) reconstructions of coronary CT angiography images (CCTA), of which the orientation is synchronized in real-time with two-dimensional (2D) invasive coronary angiograms from the same mammal comprising:
i) a device for performing invasive coronary angiography of a mammal, said device comprising at least one radiographic arm and at least one display monitor,
ii) the at least one radiographic arm comprising a position sensor, which is configured to measure the radiographic projection angles of said at least one radiographic arm in a continuous way, and further comprising a computer, which is configured to continuously read the radiographic projection angles measured by said position sensor and to publish this information; and
iii) a second independent computer comprising three-dimensional (3D) visualization software configured to visualize said CCTA 3D-reconstruction of the mammal, and computer executable instructions configured to connect to said computer to continuously read the position information of said position sensor and to simultaneously send the latter information to said 3D-visualization software,
iv) wherein the system is further configured to transfer the information obtained in feature iii) from said second independent computer to the display monitor and yielding 2D-invasive coronary angiograms shown side by side with the synchronized CCTA 3D-reconstructions on said display monitor.

According to an aspect of the invention there is provided a system to display three-dimensional (3D) reconstructions of coronary CT angiography images (CCTA), of which the orientation is synchronized in real-time with two-dimensional (2D) invasive coronary angiograms from the same patient. According to an aspect of the invention the movement of the radiographic C-arm is tracked in real-time, in order to synchronize the orientation of the three-dimensional coronary tree with the projection of the radiographic C-arm.

According to a specific embodiment, there is provided a manufacturer-independent approach. According to such an approach the sensor can be attached to any C-arm of any manufacturer, and any, which for example means from any third party/vendor/manufacturer, 3D visualization software can be used provided that an API is available or can be/is developed for said software. According to a specific embodiment, the manufacturer-independent approach was produced by attaching an external position sensor, such as for example an inertial measurement unit or IMU to the radiographic C-arm. According to an embodiment, this IMU is connected to a single board computer, such as for example a Raspberry Pi, which continuously communicates the sensor's—and therefore also the C-arm's—orientation with the 3D visualization software of the CCTA images, see for example FIGS. 4 and 8. Specific advantages of the system of the invention are an optimal angulation to visualize the coronary tree by angiography with minimal degree of overlapping and foreshortening without the need for additional administration of radiation or contrast agent and visualization of atherosclerotic plaque and its components during conventional coronary angiography. This leads to an optimized evaluation and visualization of the coronary lesions and less need of unnecessary angiographic acquisitions. In addition, the three-dimensional CCTA model can be used as a roadmap to assist during vessel wiring further reducing the need for additional contrast injection while the wire is advanced. Furthermore, guidance of PCI with CCTA is executed following the same principles as with intravascular imaging, e.g. IVUS or OCT. Preprocedural assessment starts with the evaluation of plaque characteristics, composition and extension. In presence of severe calcium burden, the operator may be prompted to perform an adequate vessel preparation with pre-dilatation and/or calcium modifying techniques. After stent implantation, post-dilatation with non-compliance balloons may help achieving better stent expansion. Subsequently, lesion length, a key parameter to achieve optimal results, is accurately determined using on the non-foreshortened CT-derived images and healthy landing zones proximal and distal to the lesion are identified. The continuous display of CCTA and invasive angiography allows also to use anatomical landmarks to visually co-register both modalities.

According to an optional embodiment, there is provided a system, wherein the CCTA 3D-reconstructions comprise plaque visualisation or other physiology-based visualisation such as for example FFRCT.

According to an optional embodiment, there is provided a system, wherein said at least one radiographic arm is a radiograph C-arm.

According to an optional embodiment, there is provided a system, wherein aid radiographic C-arm is a fixed or a flexible C-arm.

According to an optional embodiment, there is provided a system, wherein said position sensor is an inertial measurement unit (IMU).

According to an optional embodiment, there is provided a system, wherein the single board computer is a raspberry Pi.

According to an optional embodiment, there is provided a system, wherein the single board computer and the position sensor are attached on the outside of said at least one radiographic arm.

According to an optional embodiment, there is provided a system, wherein the single board computer and the position sensor are integrated into one device and are attached on the outside of said at least one radiographic arm.

According to an optional embodiment, there is provided a system, wherein:
the computer is a single board computer, which is configured to continuously read the radiographic projection angles measured by said position sensor and to publish this information through an application programming interface (API) by wireless means; and
the computer executable instructions of the second independent computer is a computer executable script, and the second independent computer comprising the computer executable script is configured to connect by wireless means to the API on said single board computer to continuously read the position information of said position sensor and to simultaneously send the latter information to said 3D-visualization software through an API integrated in said 3D-visualization software.

According to an optional embodiment, there is provided a system for diagnosing a mammal suffering from coronary artery disease.

According to a further aspect of the invention, there is provided a use of a radiographic arm in a system according to the previous aspect of the invention, wherein the radiographic arm comprises an externally attached position sensor, which measures the radiographic projection angles of said at least one radiographic arm in a continuous way, and further comprising an externally attached single board computer, which continuously reads the radiographic projection angles measured by said position sensor and publishes this information.

According to an optional embodiment, this information is published through an application programming interface (API) by wireless means.

According to a further aspect of the invention, there is provided a system to display three-dimensional (3D) reconstructions of coronary CT angiography images (CCTA), of which the orientation is synchronized in real-time with two-dimensional (2D) invasive coronary angiograms from the same mammal comprising:
i) a device for performing invasive coronary angiography of a mammal, said device comprising at least one radiographic arm and at least one display monitor,
ii) the at least one radiographic arm comprising a position sensor, which measures the radiographic projection angles of said at least one radiographic arm in a continuous way, and further comprising a single board computer, which continuously reads the radiographic projection angles measured by said position sensor and publishes this information through an application programming interface (API) by wireless means,
iii) a second independent computer comprising three-dimensional (3D) visualization software visualizing said CCTA 3D-reconstruction of the mammal, and a computer executable script which connects by wireless means to the API on said single board computer to continuously read the position information of said position sensor and simultaneously sends the latter information to said 3D-visualization software through an API integrated in said 3D-visualization software,
iv) transferring the information obtained in step iii) from said second independent computer to the display monitor and yielding 2D-invasive coronary angiograms shown side by side with the synchronized CCTA 3D-reconstructions on said display monitor.

According to an optional embodiment, there is provided a system, wherein the CCTA 3D-reconstructions comprise plaque visualisation or other physiology-based visualisation such as for example FFRCT.

According to an optional embodiment, there is provided a system, wherein said at least one radiographic arm is a radiograph C-arm.

According to an optional embodiment, there is provided a system, wherein said radiographic C-arm is a fixed or a flexible C-arm.

According to an optional embodiment, there is provided a system, wherein said position sensor is an inertial measurement unit (IMU).

According to an optional embodiment, there is provided a system, wherein the single board computer is a raspberry Pi.

According to an optional embodiment, there is provided a system, wherein the single board computer and the position sensor are attached on the outside of said at least one radiographic arm.

According to an optional embodiment, there is provided a system, wherein the single board computer and the position sensor are integrated into one device and are attached on the outside of said at least one radiographic arm.

According to an optional embodiment, there is provided a system for diagnosing a mammal suffering from coronary artery disease.

According to a further aspect of the invention, there is provided a radiographic arm comprising an externally attached position sensor, which measures the radiographic projection angles of said at least one radiographic arm in a continuous way, and further comprising an externally attached single board computer, which continuously reads the radiographic projection angles measured by said position sensor and publishes this information through an application programming interface (API) by wireless means.

### FIGURE LEGENDS

FIG. 1: CT-guided Coronary Intervention Algorithm

FIG. 2: Process of three-dimensional lumen and plaque reconstruction.

Panel A shows an 3D MIP images of a right coronary artery with diffuse calcifications. Panel B shows the 3D luminal and plaque reconstruction. And, panel C shows the color-coded vessel geometry. MIP Maximum intensity projection.

FIG. 3: Catheterization laboratory set up for online CT guidance.

The cath-lab set up comprises the addition of a synchronization hardware between the C-arm and CT software (black arrow) with the projection of the 3D CT derived geometry projected side by side to the angiographic image (white arrow).

FIG. 4: Impact of optimal projection for the evaluation of lesion length.

The left panel shows a left cranial two-dimensional projection with a lesion in the mid segment of the LAD, by QCA lesion length was 35.8 mm. Nevertheless, CT (mid panel) showed that lesion length was longer and suggested a steep right cranial projection to depict the true lesion length. The conventional angiography projection adapted based on the CT confirmed a lesion length of 45.6 mm.

LAD Left anterior descending artery. QCA quantitative coronary angiography.

FIG. 5: Prototype of measuring tool for online length assessment.

The top left panel shows a 3D reconstruction of a coronary lesion located in the proximal segment of the LAD. Top right panel shows a tomographic cross-section at the level of the marker. In the bottom a straight MPR reconstruction with the measurement tool and selection of the landing zones and lesion length. LAD Left anterior descending artery. MPR Multiplanar reconstruction.

FIG. 6: Atherosclerotic disease in apparent ‘normal’ angiographic vessel.

Panel A shows an MPR images of a LAD with a long non-calcified plaque from the ostial LAD until the takeoff the second diagonal branch. The cross-sections show positive remodeling and plaque burden of 80%. Conventional angiography shows mild disease in the proximal and mid LAD. The co-registration of CCTA and angiography triggered further invasive functional evaluation of the vessel resulting in an invasive FFR of 0.76.

CCTA Coronary computed tomography angiography. LAD Left anterior descending artery. MPR Multiplanar reconstruction. FFR Fractional flow reserve.

FIG. 7: Schematic presentation of one embodiment of the system of the invention depicting how the system synchronizes C-arm projection angles with external 3D-CT angiography. Specifically, in the flowchart 100 is a device which can perform invasive coronary angiography of a patient, the device comprising typically at least one radiographic arm (101) and a monitor (102). The radiographic arm comprises a position sensor (103) and a single board computer (104). A second independent computer unit (105) comprises 3D-visualisation software (106) which can visualize the images (107), a computer executable script (108) connects between 104 and 106, the information obtained in 105 is transferred to 102 and yields 2D-angiograms (109) which are depicted side by side with CCTA 3D-reconstructions (107).

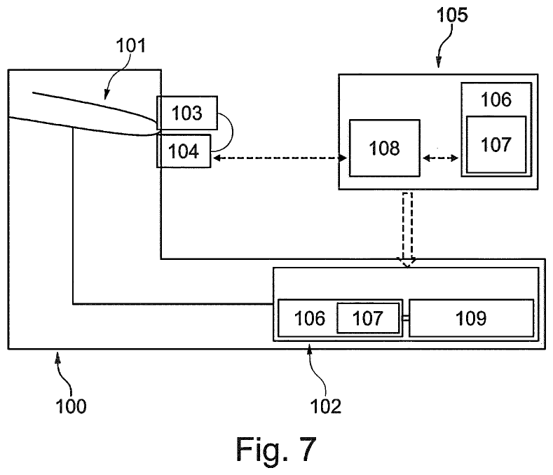

### DETAILED DESCRIPTION OF THE INVENTION

The present invention will be described with respect to particular embodiments and with reference to certain drawings, but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. The following terms or definitions are provided solely to aid in the understanding of the invention. Unless specifically defined herein, all terms used herein have the same meaning as they would to one skilled in the art of the present invention. Unless defined otherwise, all technical and scientific terms used herein have the same meaning as commonly understood by one of ordinary skill in the art (e.g. in molecular biology, interventional cardiology fluid physics, biochemistry, and/or computational biology).

Coronary CTA has become the method of choice for the evaluation of CAD. Beyond the diagnostic phase, CCTA can be used to improve patient selection for PCI, to plan and guide coronary therapeutic interventions in a fashion similar to structural heart interventions. In the present invention we have implemented the use of coronary computed tomography angiography (CCTA) in the catherization laboratory. The present inventions provide a system which adds CCTA three-dimensional images to conventional 2-D image angiography and incorporates the visualization of atherosclerotic plaque in the entire coronary tree in real-time during coronary diagnostic and therapeutic interventions. This novel approach enhances invasive procedures and can improve clinical outcomes of patients suffering from coronary artery disease (CAD).

Accordingly, the present invention provides in a first embodiment a system to display three-dimensional (3D) reconstructions of coronary CT angiography images (CCTA), of which the orientation of the images is synchronized in real-time with two-dimensional (2D) invasive coronary angiograms from the same mammal comprising:
i) a device for performing invasive coronary angiography (100) of a mammal, said device comprising at least one radiographic arm (101) and at least one display monitor (102),
ii) the at least one radiographic arm (101) comprising a position sensor (103), which measures the radiographic projection angles of said at least one radiographic arm in a continuous way, and further comprising a single board computer (104), which continuously reads the radiographic projection angles measured by said position sensor and publishes this information through an application programming interface (API) by wireless means,
iii) a second independent computer (105) comprising three-dimensional (3D) visualization software (106) visualizing said CCTA 3D-reconstruction of the mammal (107), and a computer executable script (108) which connects by wireless means to the API on said single board computer to continuously read the position information of said position sensor and simultaneously sends the latter information to said 3D-visualization software through an API integrated in said 3D-visualization software,
iv) transferring the information obtained in step iii) from said second independent computer to the display monitor (102) and yielding 2D-invasive coronary angiograms (109) shown side by side with the synchronized CCTA 3D-reconstructions (107) on said display monitor (102).

The device 100: during conventional coronary angiography a patient lies in a supine position on the table being a part of a C-arm unit. The X-ray tube moves rotationally in two perpendicular planes (horizontal and vertical, id est including the movements left, right and cranio-caudal which makes any required type of projection possible. In order to visualize coronary vessels a contrast agent is administered intravenously, a thin catheter was previously introduced into the brachial, radial or femoral artery and its movement is traced by fluoroscopy and observed on a monitor by the operator. A monitor (102) can be incorporated into the device or can be independent from the device.

In a specific aspect to illustrate this system of the invention further, in the following non-limiting example it is explained how the system performs. Typically, a patient suspected of coronary artery disease will, in a standard of care clinical setting, be diagnosed with coronary CT angiography. Subsequently, after the images are taken by means of dedicated software (such as for example Medis QAngio CT software, Leiden, Netherlands) a 3-dimensional reconstruction (typically this is accomplished offline) of the coronary tree (derived from these

CCTA images) including quantitative plaque assessment and three-dimensional complete tree plaque reconstructions is made. Optionally, atherosclerotic plaque components can be colour-coded based on Hounsfield Units (HU) (e.g. white for calcified structures with HU>320, green for plaque components between 50 and 320 HU, and red for low-attenuation plaques). The results are three-dimensional plaque portrays (also called a plaque map), which facilitates imaging interpretation. Plaque components and volume cannot be visualized using conventional coronary angiography. Optionally, the software includes a measurement tool to evaluate the length of the plaque that will impact the decision of stent size to be administered by the interventional cardiologist.

In a particular embodiment the real-time position of the C-arm is determined by using a frame grabber (for example pointing a video camera pointed on the C-arm display monitor 102), to grab in real-time the C-arm projection images (which usually show the C-arm projection angles in real-time) and use optical character recognition (OCR) to read the projection angles from the captured video.

In a next step when the patient is subjected to invasive coronary angiography (typically the patient is in the same supine position as when the patient was subjected to CCTA). A position sensor (for example a Sense HAT, which device comprises an Inertial Measurement Unit) which is connected to a single-board computer (such as for example a Raspberry Pi 4), is attached to the imaging detector of the C-arm, which arm moves the X-ray modality used for fluoroscopically guided procedures in the catheter lab. In the background the Raspberry Pi continuously queries the Inertial Measurement Unit (IMU) sensor data from the Sense HAT and publishes the data on a wireless network (Wi-Fi) through a RESTful application program interface. An independent second computer (such as a laptop) then connects via wireless means (for example via Bluetooth or WiFi) to for example the WiFi network of the Raspberry Pi and runs a computer executable script (for example written in Python or any other convenient computer language program) that continuously queries the RESTful API to read the sensor data. Finally, the computer executable script connects to a further API (such as for example a ZeroMQ API, source: Medis) to make the sensor data available for the dedicated visualization Software. The ZeroMQ API is integrated into the 3-D visualization software. It is clear, that according to alternative embodiments, instead of the wireless network and/or the API, any other suitable way for exchanging this information between the first computer (104), in the current embodiment the Raspberry Pi, and the independent second computer (105) are also possible, such as for example a wired connection, and/or any other suitable interface for exchanging the information between the first computer (104) and the second independent computer (105). It is further clear, that according to alternative embodiments, instead of a computer executable script (108), any other suitable computer executable instructions (108) are also possible. It is further clear, that according to alternative embodiments, instead of the further API of the 3-D visualization software, any suitable alternative way of making the sensor data available to the visualisation software, is also be possible.

The end result is the integration of 3D reconstruction images of the coronary anatomy (including lumen and plaque) in the catheterization laboratory. With the system of the invention both 3-dimensional CT-derived reconstructed lumen and atherosclerotic plaques are projected side by side with the two-dimensional invasive angiography image. It is important to stress the “side by side projection” since prior art processes apply superimposed or ‘co-registered’ images (for example as shown by Winck O. et al (2009) Cardiol. Clin. 27, 513-529). In the present invention the three-dimensional coronary tree is synchronized with the fluoroscopy angiography device (and with the patient table as in the prior art (see Wink et al (2009) Cardiol. Clin. 27, 513-529). Further, in the latter Wink et al reference a 3D visualization of the plaques is not shown and the approach in Wink et al only works for one manufacturer simultaneously (for example manufacturer-1 3D visualization software with manufacturer-1 cathlab combination). In the present system matched angulations between the images are displayed meaning that when the C-arm of the angio-tube rotates, on screen the 3D CT-derived reconstruction rotates and moves correspondingly.

The computer executable script for the RESTful API (an application which is freely available) was adjusted in order to convert the rotation angles coming from the IMU sensor to actual C-arm angles. A computer executable script connects the second independent computer (e.g. a laptop) to the RESTful API (Raspberry Pi) and ZeroMQ API (software originally from Medis but slightly modified). Finally, a small windows batch script on the laptop ensures that all components interact automatically and runs the computer executable scrip code automatically.

Importantly, the Raspberry Pi is in one embodiment of the system configured as a network access point, providing its own Wi-Fi network to which external devices can connect wirelessly. This brings the advantage that the laptop and the Raspberry Pi do not need any connection to a secure internal hospital (or operating rooms) network. It is clear, that according to alternative embodiments, suitable alternative single board computers (104) to the Raspberry Pi are also possible. Further, although embodiments with a single board computer are preferable, it is clear, that according to still further embodiments any suitable computer (104) is also possible.

As already mentioned above, alternative embodiments are possible in which alternative ways are possible for obtaining and providing any suitable sensor derived position data to any suitable 3-D visualisation software.

According to some embodiments the C-arm's position may be determined by attaching one or multiple external devices to the C-arm, with the goal of measuring it's orientation. According to some embodiments this device may comprise one or more of the following: an inertial measurement unit (IMU), an attitude heading reference system (AHRS) or an inertial navigation system (INS). Optionally the device may comprise even further sensors, such as for example one or more magnetometers, accelerometers, gyroscopes, etc. . According to a particular embodiment there could be made use of sensor fusion algorithms configured to combine, the data of at least two of the different sensors, such as for example two or more of the IMU, AHRS, INS, . . . into position information that represents the 3D orientation of the C-arm. This position information representing the 3D orientation of the C-arm may for example be parametrized as Euler angles, such as for example pitch, roll, yaw; quaternions; an axis-angle; a rotation matrix; a direction cosine matrix or DTM, . . . . According to such embodiments, these sensor fusion algorithms are implemented by means of computer executable instructions configured to be processed by a computer. The computer may be part of the device itself, or may be an external device, for example in case the device does not comprise a suitable computer. The 3D position information, such as for example a 3D orientation parameter, i.e. the Euler angles, quaternions, axis-angle, rotation matrix, direction cosine matrix (DTM), or any other suitable 3D orientation parameter, is then used as input parameter in 3D viewer software.

According to preferred embodiments such 3D viewer software or 3D visualisation software is capable to accept these parameters as input to orient a visualisation of a 3D object accordingly. In order to assist with aligning the position information with the coordinate system of the 3D visualisation software, according to a preferred embodiment there can be made use of a calibration sequence. The calibration sequence makes sure that a predetermined reference orientation, or “zero” orientation in the 3D visualisation software is aligned with the actual reference orientation or “zero” of the C-arm. Typically this reference orientation of the C-arm corresponds to the anterior-posterior projection or AP projection, or in other words an orientation in which left or right anterior oblique view angulations and cranial or caudal angulations are 0°, i.e. LAO/RAO 0° and CRAN/CAUD 0°. Such a calibration sequence allows to accommodate for different vendors and slight differences in how the device is attached to the C-arm. Preferably, the calibration sequence is performed during a brief period, such as for example 20 seconds or less, for example 10 seconds, as soon as the first sensor/orientation data is available shortly after connecting to the device. It is clear that during the calibration sequence, preferably the C-arm is positioned/oriented in the AP projection. According to a preferred embodiment, the average 3D orientation is calculated from the position information received during that calibration period and used as an offset value for any future measurements, according to a particular embodiment this offset value may for example be subtracted from any future measurements. According to alternative embodiments, the connection with the device for publishing the position information may be implemented by means of a wired interface, such as for example a suitable USB connection, a suitable Serial connection, . . . ; or a suitable wireless interface, such as for example WiFi, Bluetooth, Ultra WideBand, Radiofrequency, . . . . It is clear that such wired or wireless communication interfaces, according to some embodiments, may exchange any suitable data, such as for example the position information, by means of any suitable communication protocol, such as for example TTL, Serial, USB, SPI, OSC, MATT, ZeroMQ, . . . .

According to a preferred embodiment, the device comprises an IMU sensor and a computer with onboard sensor fusing algorithms, which is also referred to as an NGIMU. Preferably such an NGIMU device provides 3-D position information in format referenced by three orthogonal IMU axes. Preferably, the device is configured such that one axis of the position information is aligned, or substantially aligned, with the LAO/RAO axis, and another axis of the position information is aligned with the CRAN/CAUD axis, for example as Euler angles. According to a preferred mode of operation, for example after a connection for data exchange is established between the second independent computer (105) and the NGIMU, for example via WiFi, computer executable instructions, for example a python script, initiates a suitable connection for exchange of the position information, for example a connection with the device using the OSC protocol, and further initiates a connection with the 3D visualisation software, such as for example a connection to the CathlabViewer using the ZeroMQ API. According to a particular embodiment, the mode of operation continues by awaiting the transmission and reception 3D position information over the established connection, for example by means of an infinite while loop that waits until incoming 3D orientation data from the NGIMU parametrized as Euler angles is received. According to a particular embodiment, the mode of operation is continued with a calibration sequence, which for example during the first 10 seconds of incoming position information data, aligns this data with the actual position of the C-arm, which remains for example positioned in the AP projection, or in other words both LAO/RAO and CRAN/CAUD at zero degrees during the calibration sequence. The data received during these first 10 seconds of the calibration sequence is then for example averaged for each coordinate axis and used as an offset for the data received after the initial 10 seconds.

As, in the preferred embodiment two NGIMU's axes are aligned with the relevant axes of the C-arm, the Euler angles of these two axes respectively represent the LAO/RAO and CRAN/CAUD angulations. Each exchange of position information, such as for example each OSC message received from the NGIMU, can then be simply forwarded as LAO/RAO and CRAN/CAUD angulations to the 3D visualisation software, for example CathlabViewer, in a data format, such as for example in a ZeroMQ message.

A ‘sense HAT’ is a device which comprises several different sensors, one of these sensors is an IMU. The other sensors available on the Sense HAT, e.g. temperature and humidity sensors, are not used in the system of the invention. An IMU (Inertial Measurement Unit) is an electronic device that measures and reports a body's specific force, angular rate, and also the orientation of the body, using a combination or accelerometers, gyroscopes and optionally magnetometers. In a specific embodiment a wireless IMU is used, also known as a WIMU.

A ‘raspberry Pi’ is a low cost, credit-card sized computer that plugs into a computer monitor and uses a standard keyboard and mouse. The Raspberry Pi is a series of small single-board computers by the Raspberry Pi foundation.

A ‘RESTful API’ is an application program interface (API) that uses HTTP requests to GET, PUT, POST and DELETE data.

In yet another embodiment the system comprises at least one radiographic arm which is a radiograph C-arm.

In yet another embodiment in the system of the invention radiographic C-arm is a fixed or a mobile (or flexible) C-arm.

In a particular embodiment a fixed C-arm can be floor-mounted or ceiling-mounted. The option is chosen by the user on the space condition or operating room.

In yet another embodiment the system of the invention comprises a position sensor which is an inertial measurement unit (IMU).

In yet another embodiment the system comprises a Raspberry Pi single board computer.

In yet another embodiment the single board computer and the position sensor are attached on the outside of said at least one radiographic arm. In a specific embodiment these devices are simply attached to the radiographic arm with glue or other attachment means.

In yet another embodiment the invention provides a radiographic arm (101) comprising an externally attached position sensor (103), which measures the radiographic projection angles of said at least one radiographic arm in a continuous way, and further comprising an externally attached single board computer (104), which continuously reads the radiographic projection angles measured by said position sensor and publishes this information through an application programming interface (API) by wireless means.

In yet another embodiment the system as described herein is used for diagnosing a mammal suffering from coronary artery disease.

In the present invention a “system” is equivalent to a “device” or an “apparatus”.

A computing device, such as for example the first computer and the second independent computer as used herein, is generally representative of any device suitable for performing the processing and analysis techniques discussed within the present disclosure. In some embodiments, the computing device includes a processor, random access memory, and a storage medium. In that regard, in some particular instances the computing device is programmed to execute steps associated with the data acquisition and analysis described herein. Accordingly, it is understood that any steps related to data acquisition, data processing, presentation of 3D-images of CCTA, instrument control, and/or other processing or control aspects of the present disclosure may be implemented by the computing device using corresponding instructions stored on or in a non-transitory computer readable medium accessible instances, by the computing device. In some the computing device is a console device. In some instances, the computing device is portable (e.g. handheld, on a rolling cart, etc.). Further, it is understood that in some instances the computing device comprises a plurality of computing devices. In that regard, it is particularly understood that the different processing and/or control aspects of the present disclosure may be implemented separately or within predefined groupings using a plurality of computing devices. Any divisions and/or combinations of the processing and/or control aspects described herein across multiple computing devices are within the scope of the present disclosure.

It is understood that any communication pathway between the position sensor, single board computer and the second independent computing device may be utilized, including physical connections (including electrical, optical, and/or fluid connections), wireless connections, and/or combinations thereof. In that regard, it is understood that the connection is wireless in some instances. In some instances, the connection is a communication link over a network (e.g. intranet, internet, telecommunications network, and/or other network). In that regard, it is understood that the second independent computing device is positioned remote from an operating area where the C-arm is being used in some instances. Having the connection include a connection over a network can facilitate communication between the C-arm and the remote computing device regardless of whether the computing device is in an adjacent room, an adjacent building, or in a different state/country. Further, it is understood that the communication pathway between the single board computer and the second independent computing device is a secure connection in some instances. Further still, it is understood that, in some instances, the data communicated over one or more portions of the communication pathway between the single board computer and the second independent computing device is encrypted.

It is also understood that the information obtained regarding characteristics of the coronary artery disease (predicted to be diffuse intermediate or focal lesion, predicted to be a lesion of certain length and size) can be compared with or considered in addition to other representations of the lesion or stenosis and/or the vessel (e.g. IVUS (including virtual histology), OCT, ICE, Thermal, Infrared, flow, Doppler flow, and/or other vessel data-gathering modalities) to provide a more complete and/or accurate understanding of the vessel characteristics. For example, in some instances the information regarding characteristics of the lesion or stenosis and/or the vessel as obtained by the system of the invention are utilized to confirm information calculated or determined using one or more other vessel data-gathering modalities.

It is to be understood that although particular embodiments, specific configurations as well as materials and/or molecules, have been discussed herein for methods according to the present invention, various changes or modifications in form and detail may be made without departing from the scope and spirit of this invention. The following examples are provided to better illustrate particular embodiments, and they should not be considered limiting the application. The application is limited only by the claims.

### EXAMPLES

### 1.CT based Evaluation for Revascularization Procedures

### Luminal Assessment

Coronary CTA overcomes a frequent problem of invasive angiography i.e. vessel foreshortening. (4) CT in the planning phase of a coronary intervention aids in providing the best angiographic projection in the catheterization laboratory, thereby minimizing foreshortening and overlapping of the segment of interest. This becomes even more relevant in the evaluation of bifurcation lesions where the visualization of the side-branch ostium is often sub-optimal with conventional angiography. (5) Furthermore, angiographic foreshortening observed in two-dimensional projection images impairs accurate evaluation of lesion length. (4) This is a frequent cause of incomplete plaque coverage and geographical miss; these latter issues are associated with adverse events after stent implantation. (6) In addition, CT-derived lesion length evaluation incorporates the atherosclerotic extension that is not visible with conventional angiography; this approach mimics the evaluation obtained by intravascular imaging techniques (e.g. IVUS and optical coherence tomography).

The two most relevant metrics derived from CCTA analysis are minimal lumen diameter (MLD) and reference vessel diameter (RVD). CT-derived quantitative coronary analysis (QCA) have been shown to have a high degree of agreement with the true luminal dimensions. (7) In the clinical setting, MLD is used to define lesion severity, whereas for percutaneous revascularization planning, RVD distal to the lesion is used to select stent diameter. CT based quantitative coronary analysis (QCA) have been shown to have a very high agreement with respect to luminal dimensions from conventional angiography, and diameter measurements based on angiographic (conventional angiography and CCTA) have shown to be smaller compared to intravascular ultrasound (IVUS). (8) The systematic differences in vessel dimensions between these methods are partially explained by the differences in spatial resolution and the physical properties of the devices. For PCI planning, CT-derived RVD, obtained at healthy coronary segments, can be incorporated in the decision process concerning stent diameter selection.

### Plaque Assessment

In addition to the lumen, CCTA allows to visualize the atherosclerotic plaque and determine its burden. (9,10) Plaques can be qualitative and quantitively characterized. It has been shown that CCTA helps identify high risk plaques using measures of remodeling (remodeling index) and also allow the characterization of lesions through their Hounsfield units (HU) and appearances. Calcified plaques can be visualized as white structures with high HU, and their burden and circumference can be evaluated. (11) High calcium burden is associated with lower stent expansion and higher rates of adverse events after PCI. (12) (13) Hence, visualization of high calcium burden in the planning phase of coronary intervention may prompt use of calcium modification techniques (e.g. rotational atherectomy, orbital atherectomy, excimer laser or intravascular lithoplasty) to facilitate stent expansion. (14-16)

Stent expansion, which depends partially on the underlying plaque, is an independent predictor of major adverse events after PCI. In the other side of the plaque spectrum, plaques with low HU, the so-called soft-plaques (i.e. HU<50) have been identified as independent predictors of acute coronary syndromes, peri-procedural myocardial infarction and no-reflow phenomenon. (1) (17) The assessment of plaque extension with CCTA allows to extrapolate the normal-to-normal concept described with intravascular imaging. (18) By ‘landing’ the stents on healthy coronary segments, plaque coverage is assured reducing the risk of restenosis and thrombosis.

Quantitative plaque assessment forms the basis of the three-dimensional plaque reconstructions used during online CT guidance (discussed below). Atherosclerotic plaque components are color-coded based on HU (e.g. white for calcified structures with HU>320, green for plaque components between 50 and 320 HU, and red for low-attenuation plaques). This result in three-dimensional plaque portrays, a plaque map, that facilitates imaging interpretation, the evaluation of disease extension, volume and composition. Simultaneous plaque visualization during conventional angiography optimizes the interpretation of the images given that apparent ‘normal’ segments may be diffusely disease. It should be highlighted that the interpretation of calcified plaques demands a special consideration given the overestimation of calcium volume due to blooming artefacts.

### Functional Assessment

Using CCTA, 3D coronary geometries can be extracted and used to perform fluid dynamic simulations. Assuming a normal response of the coronary microcirculation to hyperemic stimulus, and adjusting microvascular resistance to vessel specific myocardial mass, blood flow simulations can estimate coronary pressures enabling to compute fractional flow reserve, the ratio of distal coronary pressure and aortic pressure during hyperemia. Clinical trials have demonstrated an improved diagnostic performance of FFR_{CT} compared to a visual assessment for the detection of hemodynamically significant lesions. A detailed description of the principles, the potential and the outcome data are beyond the scope of this review. In the next section we describe the usefulness of FFR_{CT} for patient selection for PCI.

### 2.CCTA Derived 3-D Reconstruction as a Guide in the Catheterization Laboratory

### Precise PCI and Procedural Planning (P4) Algorithm

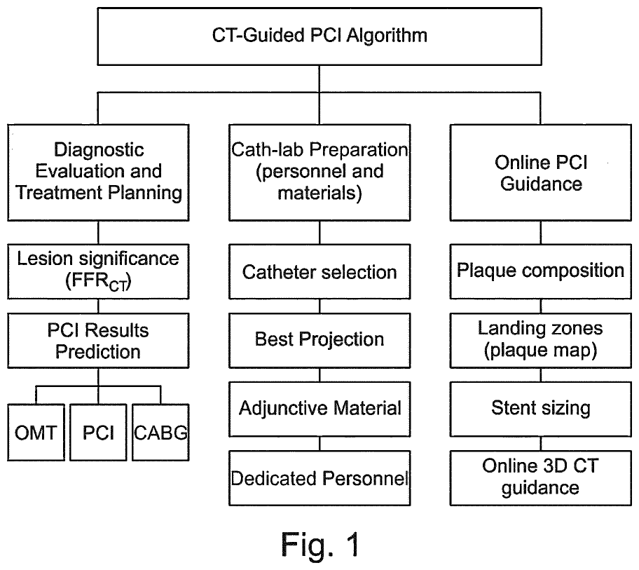

The P4 algorithm is shown in FIG. 1. Based on three mainstays namely diagnostic evaluation, catheterization laboratory preparation and online guidance, the algorithm proposes the incorporation of CT into several phases of the management of patients with obstructive CAD.

### Diagnostic Evaluation

The initial part of the algorithm extends the evaluation of the hemodynamic significance of CAD in two domains: (1) Determination of the pattern of CAD (i.e. focal or diffuse) and (2) the prediction of PCI results. Functional evaluation with FFR_{CT} characterizes the pathophysiological pattern (e.g. focal or diffuse) of CAD non-invasively. (19) This can be visualized either on the color-coded geometry or by a virtual FFR_{CT} pullback curve. (20) In cases of focal functional disease, pressure losses are circumscribed to anatomical stenoses (i.e. lesion specific ischemia), this vessel phenotype is favorable for PCI in terms of post-intervention vessel physiology. In contrast, cases of diffuse functional CAD show no focal pressure drops, these vessels often exhibit diffuse atherosclerosis on CCTA, and despite the presence of one or several narrowing, PCI results are sub-optimal in terms of post-PCI FFR.

Therefore, the evaluation of the anatomic and physiological pattern of CAD aids predicting the likelihood of functional complete revascularization and relieve from angina. (20) (21)

### Catheterization Laboratory Preparation

From the evaluation of the position of the coronary ostia to the planning of complex PCI, a non-invasive stratification of the complexity of CAD aids on the organization of the catheterization laboratory. Although relative uncommon, identification of coronary anomalies informs upon the more adequate type of coronary catheters and cannulation technique. (22) Moreover, CAD in the coronary ostia may change the cannulation strategy avoiding deep vessel catheterization that may conceal an obstructive lesion and flaw invasive FFR measurement. Furthermore, in patients with graft anastomosis in the aorta, CCTA aids in localizing conduits and expediting cannulation. In these scenarios, a CT-guided approach saves time, contrast, and reduces unnecessary radiation exposure.

Based on CCTA, complex interventions can be better planned in the catheterization laboratory. In case of chronic coronary occlusions, CCTA helps to better identify the distal vessel and route for PCI than invasive angiography and predicts guidewire crossing and procedural success. (23) The CT RECTOR and J-CTO score has been shown to be helpful to predict the likelihood of success of CTO intervention integrating a number of CT variables allowing the planning of intervention in advance of angiography. (24) Measures such as occlusion length, stump morphology, angulation, cross sectional area of calcification, and the outlet morphology can all be routinely obtained from CCTA and help inform the likelihood of CTO recanalization. (25) Beyond chronic total occlusions, in other challenging lesion subsets such as left main disease, bifurcations or severely calcified coronary vessels CCTA can inform upfront on the need for dedicated devices to increase the likelihood of success.

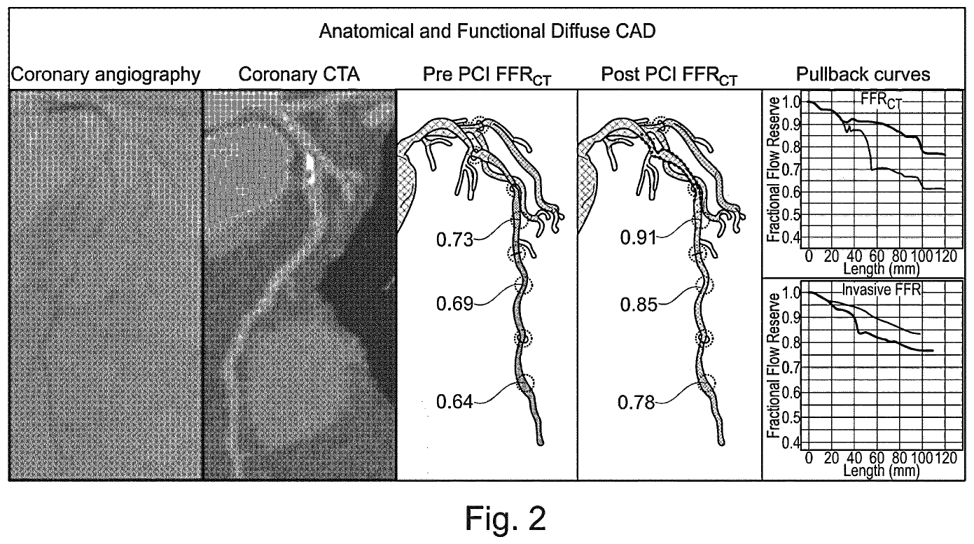

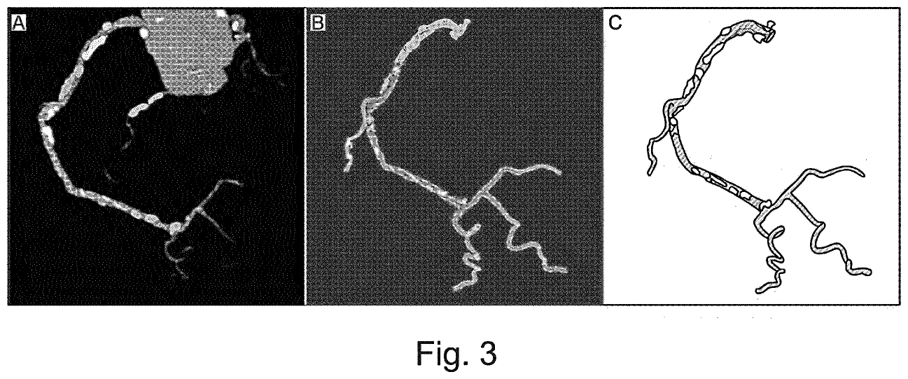

Online procedural and PCI guidance The visualization of the coronary circulation derived from CCTA provides a three-dimensional view of the coronary tree and the plaque component during conventional angiography procedures. Both lumen and atherosclerotic plaques are reconstructed and projected side by side with the two-dimensional invasive angiography. To facilitate online interpretation, plaques components are color-coded based on their Hounsfield units. The process of coronary vessel reconstruction is shown in FIG. 2. During the diagnostic procedure, the right and left coronary artery are displayed sequentially in coordination with the invasive catheterization. The movement of the C-arm is tracked in real-time, in order to synchronize the orientation of the three-dimensional coronary tree with the projection of the fluoroscopic C-arm. A manufacturer-independent approach was accomplished by attaching an external sensor, called an inertial measurement unit (IMU) to the C-arm (such a manufacturer independent approach means that 1) the sensor can be attached to any C-arm of any manufacturer and that any (from any third party/vendor/manufacturer) 3D visualization software can be used provided that an API is available or can be/is developed for said software). The IMU is connected to a Raspberry Pi, which continuously communicates the sensor'—and therefore also the C-arm's—orientation with the 3D visualization software (FIG. 3).

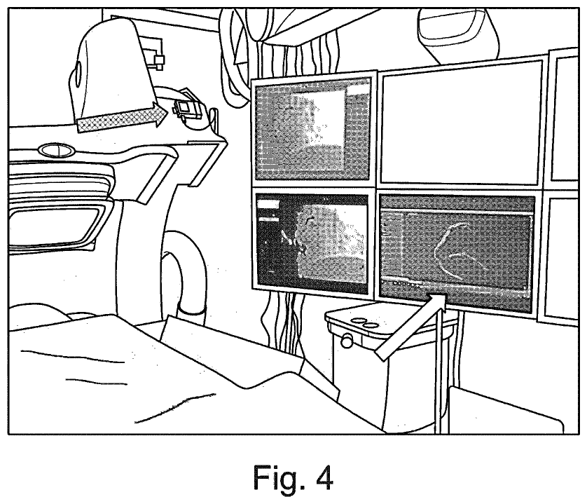

During the procedure, the coronary anatomy derived from CCTA is continuously projected during changes in angiographic projections. At each projection, it's possible to assess the degree of overlapping and foreshortening without additional radiation or contrast. FIG. 4 shows a case example on the impact of patient-specific projection on lesion length assessment. Tailored angulations optimize lesion evaluation and prevents unnecessary angiographic acquisitions. Furthermore, the three-dimensional CCTA model can be used as a 3D roadmap to assist during vessel wiring further reducing the need for additional contrast injection while the wire is advanced.

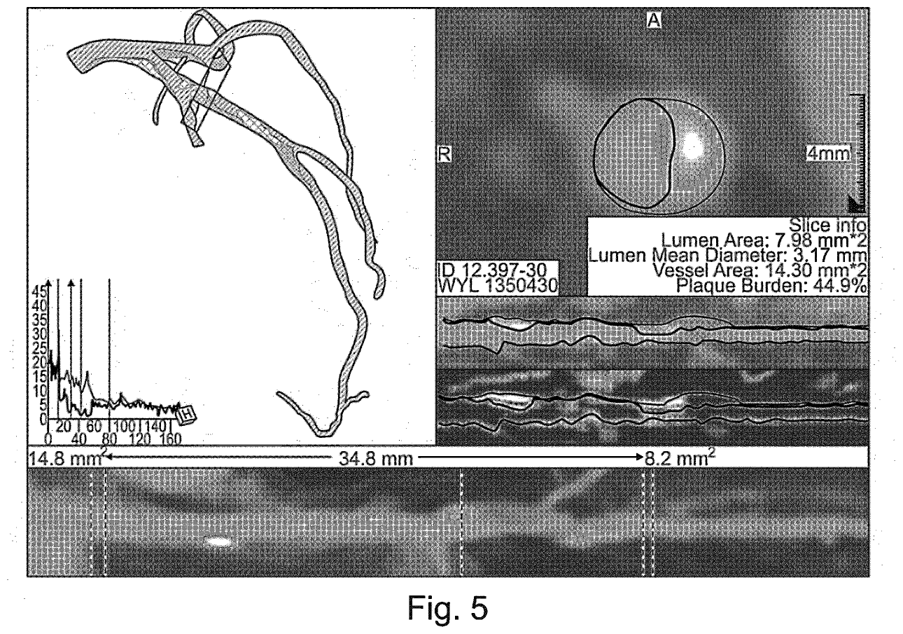

Guidance of PCI with CCTA follows the same principles as with intravascular imaging (e.g. IVUS or OCT). (18) Preprocedural assessment starts with the evaluation of plaque characteristics, composition and extension. Lesion length is determined based on healthy landing zones proximal and distal to the lesion (FIG. 5). The continuous display of CCTA and invasive angiography allows also the use of anatomical landmarks to visually co-register both modalities.

### 3.Clinical Implications

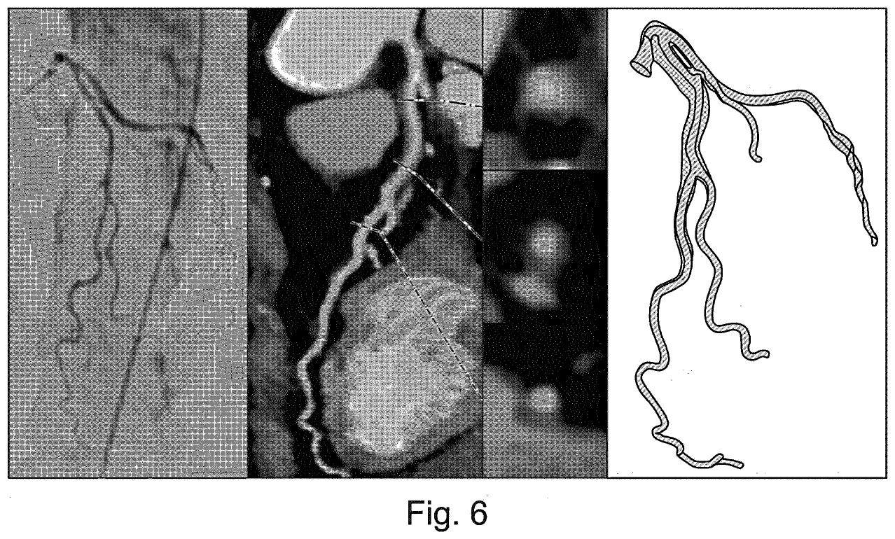

Coronary CTA is emerging as the preferred method for non-invasive assessment of CAD. Consequently, the number of patients referred for an invasive angiography with a CCTA is expected to increase. (26) Clinical decision based on the morphological and functional component may translate in better selection of patients for percutaneous revascularization in a fashion similar to the way CTA has been used to better inform and plan structural heart disease interventions. Likewise, a preprocedural stratification of case complexity may help to better organize timeslots and personnel, and in this way improving the catheterization laboratory workflow, efficiency and resource utilization. The integration of CCTA images in the catheterization laboratory is also likely to improve the safety of the procedure in terms of radiation dose and contrast volume. Moreover, the visualization of atherosclerotic disease in apparent ‘normal’ or mild disease angiographic segments might increase the use of invasive functional and imaging assessment (FIG. 6). Altogether, the integration of CCTA in the cath-lab has the potential to improve the diagnostic performance of conventional angiography and patient management. During PCI, CCTA provides a ‘live’ IVUS-like imaging of the atherosclerotic plaque. The optimization of the angiographic information with plaque visualization is likely to be translated in improved PCI technique with complete plaque coverage and might improve clinical outcomes after PCI. Nonetheless, it should be highlighted that after stent implantation IVUS or OCT are the preferred methods to assess stent expansion and apposition.

### REFERENCES

1. Motoyama S, Sarai M, Harigaya H et al. Computed tomographic angiography characteristics of atherosclerotic plaques subsequently resulting in acute coronary syndrome. Journal of the American College of Cardiology 2009;54:49-57.
2. Newby D E, Adamson P D, Berry C et al. Coronary CT Angiography and 5-Year Risk of Myocardial Infarction. The New England journal of medicine 2018;379:924-933.
3. Knuuti J, Wijns W, Saraste A et al. 2019 ESC Guidelines for the diagnosis and management of chronic coronary syndromes: The Task Force for the diagnosis and management of chronic coronary syndromes of the European Society of Cardiology (ESC). European heart journal 2019.
4. Collet C, Grundeken M J, Asano T, Onuma Y, Wijns W, Serruys P W. State of the art: coronary angiography. Eurolntervention: journal of EuroPCR in collaboration with the Working Group on Interventional Cardiology of the European Society of Cardiology 2017;13:634-643.
5. Collet C, Onuma Y, Cavalcante R et al. Quantitative angiography methods for bifurcation lesions: a consensus statement update from the European Bifurcation Club. Eurolntervention : journal of EuroPCR in collaboration with the Working Group on Interventional Cardiology of the European Society of Cardiology 2017;13:115-123.
6. Campbell P T, Mahmud E, Marshall J J. Interoperator and intraoperator (in)accuracy of stent selection based on visual estimation. Catheter Cardiovasc Intent 2015;86:1177-83.
7. Collet C, Onuma Y, Grundeken M J et al. In vitro validation of coronary CT angiography for the evaluation of complex lesions. Eurolntervention : journal of EuroPCR in collaboration with the Working Group on Interventional Cardiology of the European Society of Cardiology 2018;13:e1823-e1830.
8. Collet C, Chevalier B, Cequier A et al. Diagnostic Accuracy of Coronary CT Angiography for the Evaluation of Bioresorbable Vascular Scaffolds. JACC Cardiovascular imaging 2018;11:722-732.
9. van Rosendael A R, Al′Aref SJ, Dwivedi A et al. Quantitative Evaluation of High-Risk Coronary Plaque by Coronary CTA and Subsequent Acute Coronary Events. JACC Cardiovascular imaging 2019;12:1568-1571.
10. Conte E, Mushtaq S, Pontone G et al. Plaque quantification by coronary computed tomography angiography using intravascular ultrasound as a reference standard: a comparison between standard and last generation computed tomography scanners. Eur Heart J Cardiovasc Imaging 2020;21:191-201.
11. Dettmer M, Glaser-Gallion N, Stolzmann P et al. Quantification of coronary artery stenosis with high-resolution CT in comparison with histopathology in an ex vivo study. EurJ Radiol 2013;82:264-9.
12. Fujino A, Mintz G S, Matsumura M et al. A new optical coherence tomography-based calcium scoring system to predict stent underexpansion. Eurolntervention : journal of EuroPCR in collaboration with the Working Group on Interventional Cardiology of the European Society of Cardiology 2018;13:e2182-e2189.
13. Genereux P, Madhavan M V, Mintz G S et al. Ischemic outcomes after coronary intervention of calcified vessels in acute coronary syndromes. Pooled analysis from the HORIZONS-AMI (Harmonizing Outcomes With Revascularization and Stents in Acute Myocardial Infarction) and ACUITY (Acute Catheterization and Urgent Intervention Triage Strategy) TRIALS. Journal of the American College of Cardiology 2014;63:1845-54.
14. Ali Z A, Brinton T J, Hill J M et al. Optical Coherence Tomography Characterization of Coronary Lithoplasty for Treatment of Calcified Lesions: First Description. JACC Cardiovascular imaging 2017;10:897-906.
15. Fujino A, Mintz G S, Lee T et al. Predictors of Calcium Fracture Derived From Balloon Angioplasty and its Effect on Stent Expansion Assessed by Optical Coherence Tomography. JACC Cardiovascular interventions 2018;11:1015-1017.
16. Kobayashi N, Ito Y, Yamawaki M et al. Optical frequency-domain imaging findings to predict good stent expansion after rotational atherectomy for severely calcified coronary lesions. The international journal of cardiovascular imaging 2018;34:867-874.
17. Uetani T, Amano T, Kunimura A et al. The association between plaque characterization by CT angiography and post-procedural myocardial infarction in patients with elective stent implantation. JACC Cardiovascular imaging 2010;3:19-28.
18. Maehara A, Matsumura M, Ali Z A, Mintz G S, Stone G W. IVUS-Guided Versus OCT-Guided Coronary Stent Implantation: A Critical Appraisal. JACC Cardiovascular imaging 2017;10:1487-1503.
19. Mizukami T, Tanaka K, Sonck J et al. Evaluation of epicardial coronary resistance using computed tomography angiography: A Proof Concept. J Cardiovasc Comput Tomogr 2019.
20. Collet C, Sonck J, Vandeloo B et al. Measurement of Hyperemic Pullback Pressure Gradients to Characterize Patterns of Coronary Atherosclerosis. Journal of the American College of Cardiology 2019;74:1772-1784.
21. Fournier S, Ciccarelli G, Toth G G et al. Association of Improvement in Fractional Flow Reserve With Outcomes, Including Symptomatic Relief, After Percutaneous Coronary Intervention. JAMA cardiology 2019;4:370-374.
22. Grani C, Buechel R R, Kaufmann P A, Kwong R Y. Multimodality Imaging in Individuals With Anomalous Coronary Arteries. JACC Cardiovascular imaging 2017;10:471-481.
23. Mollet N R, Hoye A, Lemos P A et al. Value of preprocedure multislice computed tomographic coronary angiography to predict the outcome of percutaneous recanalization of chronic total occlusions. The American journal of cardiology 2005;95:240-3.
24. Fujino A, Otsuji S, Hasegawa K et al. Accuracy of J-CTO Score Derived From Computed Tomography Versus Angiography to Predict Successful Percutaneous Coronary Intervention. JACC Cardiovascular imaging 2018;11:209-217.
25. Opolski M P, Achenbach S, Schuhback A et al. Coronary computed tomographic prediction rule for time-efficient guidewire crossing through chronic total occlusion: insights from the CT-RECTOR multicenter registry (Computed Tomography Registry of Chronic Total Occlusion Revascularization). JACC Cardiovascular interventions 2015;8:257-267.
26. Moss A J, Williams M C, Newby D E, Nicol E D. The Updated NICE Guidelines: Cardiac CT as the First-Line Test for Coronary Artery Disease. Curr Cardiovasc Imaging Rep 2017;10:15.

1. A system configured to display three-dimensional (3D) reconstructions of coronary CT angiography images (CCTA), of which the orientation is synchronized in real-time with two-dimensional (2D) invasive coronary angiograms from the same mammal comprising:
i) a device for performing invasive coronary angiography (100) of a -mammal, said device comprising at least one radiographic arm (101) and at least one display monitor (102),
ii) the at least one radiographic arm (101) comprising a position sensor (103), which is configured to measure the radiographic projection angles of said at least one radiographic arm in a continuous way, and further comprising a computer (104), which is configured to continuously read the radiographic projection angles measured by said position sensor and to publish this information; and
iii) a second independent computer (105) comprising three-dimensional (3D) visualization software (106) configured to visualize said CCTA 3D-reconstruction of the mammal (107), and computer executable instructions (108) configured to connect to said computer (104) to continuously read the position information of said position sensor and to simultaneously send the latter information to said 3D-visualization software,
iv) wherein the system is further configured to transfer the information obtained in feature iii) from said second independent computer (105) to the display monitor (102) and yielding 2D-invasive coronary angiograms (109) shown side by side with the synchronized CCTA 3D-reconstructions (107) on said display monitor (102).

2. The system according to claim 1, wherein the CCTA 3D-reconstructions comprise plaque visualisation or other physiology-based visualisation such as for example FFR_{CT}.

3. The system according to claim 1, wherein said at least one radiographic arm is a radiograph C-arm.

4. The system according to claim 3, wherein said radiographic C-arm is a fixed or a flexible C-arm.

5. The system according to claim 1, wherein said position sensor is an inertial measurement unit (IMU).

6. The system according to claim 1, wherein the single board computer is a raspberry Pi.

7. The system according to claim 1, wherein the single board computer and the position sensor are attached on the outside of said at least one radiographic arm.

8. The system according to claim 7, wherein the single board computer and the position sensor are integrated into one device and are attached on the outside of said at least one radiographic arm.

9. The system according claim 1, wherein:
the computer (104) is a single board computer (104), which is configured to continuously read the radiographic projection angles measured by said position sensor and to publish this information through an application programming interface (API) by wireless means; and
the computer executable instructions (108) of the second independent computer (105) is a computer executable script (108), and the second independent computer (105) comprising the computer executable script (108) is configured to connect by wireless means to the API on said single board computer to continuously read the position information of said position sensor and to simultaneously send the latter information to said 3D-visualization software through an API integrated in said 3D-visualization software.

10. A method of diagnosing a mammal suffering from coronary artery disease comprising using the system of claim 1.

11. A method of using a radiographic arm (101) in a system according to claim 1, wherein the radiographic arm (101) comprises an externally attached position sensor (103), which measures the radiographic projection angles of said at least one radiographic arm in a continuous way, and further comprising an externally attached single board computer (104), which continuously reads the radiographic projection angles measured by said position sensor and publishes this information.

12. The method according to claim 11, wherein said information is published through an application programming interface (API) by wireless means.
